(19) United States
(12) Patent Application Publication
(10) Pub. No.: US 2026/0295931 A1
(43) Pub. Date: Oct. 1, 2026

(54) BIOPRINTING OF HIERARCHICALLY BRANCHING MICROVASCULAR NETWORKS

(71) Applicant: Ohio State Innovation Foundation, Columbus, OH (US)

(72) Inventors: Ryan HOOPER, Columbus, OH (US); David DEAN, Columbus, OH (US); Ciro RODRIGUEZ, Columbus, OH (US); Javier Vazquez ARMENDARIZ, Columbus, OH (US)

(21) Appl. No.: 19/489,787

(22) PCT Filed: Jun. 6, 2024
(86) PCT No.: PCT/US2024/032760

§ 371 Date: Dec. 3, 2025

Related U.S. Application Data
(60) Provisional application No. 63/506,507, filed on Jun. 6, 2023.
(60) Provisional application No. 63/604,956, filed on Dec. 1, 2023.

(51) Int. Cl. B29C 64/112; B29C 64/209; B33Y 30/00; B33Y 40/20; B33Y 70/10; B33Y 80/00; C12N 5/071

(52) U.S. Cl. CPC B29C 64/112; B29C 64/209; B33Y 30/00; B33Y 40/20; B33Y 70/10; B33Y 80/00; C12N 5/0691

(57) ABSTRACT

A challenge to the clinical viability of large-scale tissue engineering is being able to perfuse entire constructs with blood supply that persists long-term. To accomplish this, tissue engineering needs to replicate the hierarchically branching networks of microvasculature that distribute homogeneous blood supply from arteries to capillary beds in natural tissues. Chaotic Printing can be adapted to extruding wide sheets of hydrogel containing hierarchically branching layers of multiple cell types that are native to vascular tissue. An in vitro “prevascularization” period can allow these cells to form into hollow tubular structures that mimic native microvasculature and can be perfused with blood supply. These prevascularized sheets can then be implanted in a subject to integrate with native arteries and veins and perfuse injured tissue with new blood supply.

### CROSS-REFERENCE TO RELATED APPLICATIONS

This application claims priority to U.S. Provisional Patent Application No. 63/506,507, filed Jun. 6, 2023, entitled “BIOPRINTING OF HIERARCHICALLY BRANCHING MICROVASCULAR NETWORKS” and U.S. Provisional Patent Application No. 63/604,956, filed Dec. 1, 2023, entitled “BIOPRINTING OF HIERARCHICALLY BRANCHING MICROVASCULAR NETWORKS,” the respective disclosures are each expressly incorporated herein by reference.

### BACKGROUND

Bioprinting (i.e., printing structures using viable cells, biomaterials, and biomolecules) is a technique that could be used to generate alternatives to naturally derived tissue grafts, and even produce entire functional organs to combat worldwide donor shortages. Moreover, bioprinted tissue constructs can be used as modeling tools for drug screening and research. However, there are barriers to bioprinted constructs translating to the clinic, particularly with regard to the problem of vascularization. In almost all bodily tissues, cells must be within 200 μm of a nearby capillary to allow sufficient nutrient delivery and waste removal for long-term survival. This is a reason why autologous free flap grafting is commonly combined with bone or other grafted tissue during reconstructive surgical procedures. With this technique, autologous tissue can be sectioned and transferred to a new bodily region while retaining functional vasculature, either within the primary tissue or quickly provided by highly microvascular secondary tissue (e.g., muscle or fat). Autologous free flap grafting allows for immediate blood perfusion upon implantation but inherently involves considerable comorbidity and often pain at the donor site. However, the field's inability to fabricate an artificial microvascular graft is a technological need that remains unsatisfied.

### SUMMARY

A challenge to the clinical viability of large-scale tissue engineering is being able to perfuse entire constructs with blood supply that persists long-term. To accomplish this, tissue engineering needs to replicate the hierarchically branching networks of microvasculature that distribute homogeneous blood supply from arteries to capillary beds in natural tissues. Chaotic Printing can be adapted to extruding wide sheets of hydrogel containing hierarchically branching layers of multiple cell types that are native to vascular tissue. An in vitro “prevascularization” period can allow these cells to form into hollow tubular structures that mimic native microvasculature and can be perfused with blood supply. These prevascularized sheets can then be implanted in a subject to integrate with native arteries and veins and perfuse injured tissue with new blood supply.

In accordance with an aspect of the disclosure, a printing device includes a plurality of input valves that receive print material; a plurality of Kenics Static Mixer (KSM) printheads that each receive the print material from the plurality of input valves, each of the KSM printheads having a predetermined number of mixing elements that divide received print material into layers; and a multi-way valve that directs a flow pattern to a fanning nozzle outlet. The fanning output nozzle outputs a construct as a sheet.

In accordance with another aspect of the disclosure, a method of printing a micropatterned construct is disclosed. The method includes pumping a plurality of bioinks to respective valves; using the valves to control which of the plurality of bioinks is provided to kenics static mixing (KSM) printheads; mixing the output of the KSM printheads; providing mixed output of the KSM printheads to a fanning nozzle; and extruding a hydrogel sheet construct from the fanning nozzle.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIGS. 1A-1C illustrate an example Chaotic Printing printhead that uses a Kenics Static Mixer (KSM) design;

FIG. 2 illustrates an example multiplexer;

FIGS. 3A-3C illustrate an example Microvascular Appendage Sheet (MAS) printing system;

FIGS. 4-7 illustrate aspects of an example Continuously Extruded Variable Internal Channeling CEVIC device;

FIGS. 8A-8E show example 3D printed components of the CEVIC device;

FIGS. 9A-9B illustrate example valves that may be used in the CEVIC device;

FIG. 10 illustrates additional details of the example CEVIC device of FIG. 7;

FIGS. 11 and 12 show example hydrogel constructs produced by the CEVIC device of FIGS. 7 and 10;

FIG. 13 shows a measured channel thicknesses of the hydrogel constructs were relatively close to theoretical thicknesses; and

FIG. 14 illustrate viscosity measurements across varying shear rates for the hydrogel inks; and

FIGS. 15A-15D show calcein AM fluorescent microscopy images that prove the HUVECs survived the CEVIC device novel printing process and maintained viability in the hydrogel ink formulation throughout the 1-week culture period; and

FIG. 15E illustrates that CD31 protein expression in the constructs was observed at day 7 with a fluorescently conjugated CD31 antibody.

### DETAILED DESCRIPTION

### Introduction to Vascularization and Bioprinting

To accurately replicate microvasculature and other complex tissues, bioprinting may need to create layers as thin as a single cell. Extrusion bioprinting (i.e., driving cell-laden liquid hydrogel through a nozzle to be solidified upon deposition) is a relatively low cost method that could provide scalable tissue and organ bioprinting; however, the cell-laden hydrogel filaments resulting from traditional extrusion bioprinting have the lowest resolution of all bioprinting modalities (~100 μm). Light-assisted bioprinting (i.e., inducing hydrogel crosslinking via laser or projected image) reaches higher resolutions but often requires significant ultraviolet (UV) light exposure, introducing well-documented risk of DNA damage and cancer. Some light-assisted bioprinting techniques utilize visible light to address these concerns, however this requires stronger light sources to provide the same print times and curing quality as UV-based methods. Additionally, researchers of both UV and visible light bioprinting technologies are challenged with developing well-performing photoinitiators that are non-toxic in cell-laden hydrogel formulations.

In addition to achieving high resolutions, bioprinting often needs to incorporate multiple materials and cell types to replicate the highly structured spatial patterning of different cell types found in native, functional tissues. Multi-material bioprinting has been explored to incorporate multiple materials and cell types. Extrusion bioprinting of cell-laden hydrogel filaments is a common multi-material bioprinting strategy, having both single-nozzle and multi-nozzle devices. While multi-nozzle devices avoid material cross-contamination, using a single nozzle allows for one to switch material without stopping the extrusion process, an event that impacts the construct's structural integrity. Multi-material bioprinting has also been applied to handheld devices that allow for efficient, in situ deposition of fragile tissue constructs directly onto wound sites.

### Chaotic Printing

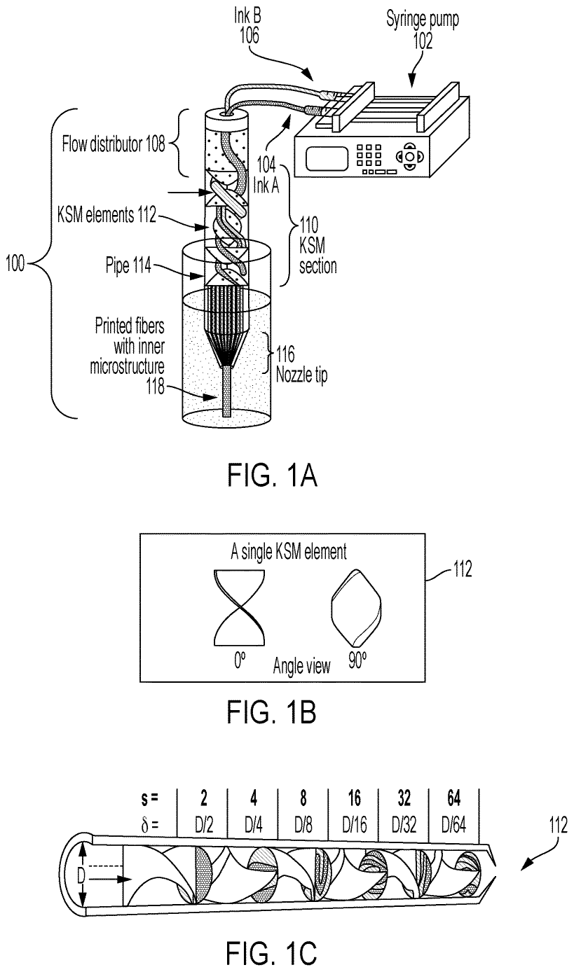

Chaotic Printing is one implementation of an extrusion-based bioprinting technology. FIGS. 1A-1C illustrate an example Chaotic Printing printhead 100 that uses a Kenics Static Mixer (KSM) 110 to successively bifurcate two hydrogel inputs 104, 106 into adjacent alternating layers that are extruded though a pipe 114 and nozzle tip 116 within a single fiber 118. The input hydrogels 104, 106 are supplied under pressure by a pump 102. The number of layers within the fiber corresponds to the number of KSM mixing elements 112 within the printhead 100. This provides precise control over the layer thickness extruded by the printhead up to a resolution significantly higher than existing state-of-the-art extrusion-based bioprinters (i.e., <10 microns versus >100 microns). Thus, Chaotic Printing is an example of extrusion-based microvascular fabrication strategy to bioprint at a high resolution.

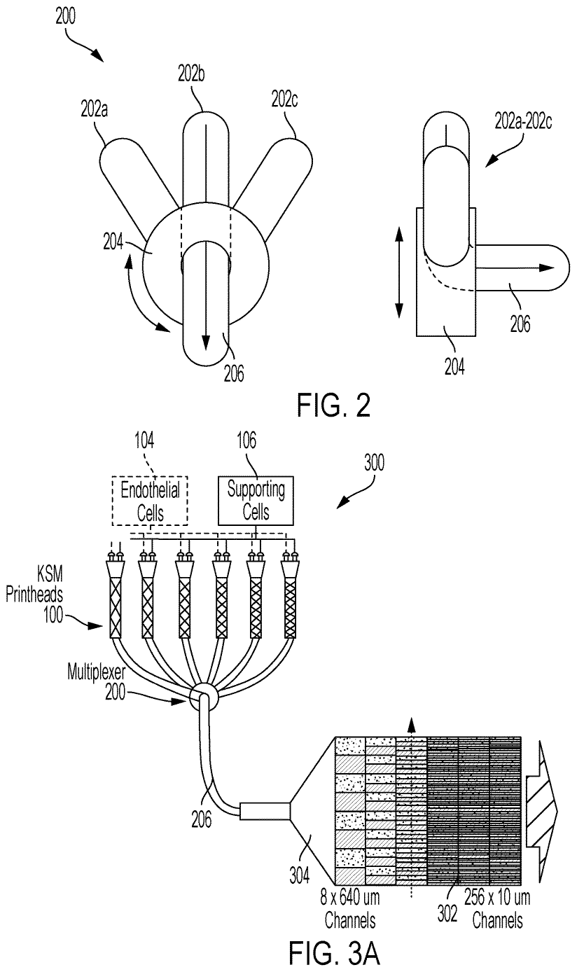

FIG. 2 illustrates an example multiplexer 200. By using a “multiplexer” to vary the number of KSM elements the hydrogel inputs travel through during one extrusion, a continuous gradient of decreasing layer thickness and increasing layer number can be fabricated within a single construct. The circular device 204 rotates to select between multiple flow inputs 202a, 202b, 202c to output hydrogel through a flow output 206.

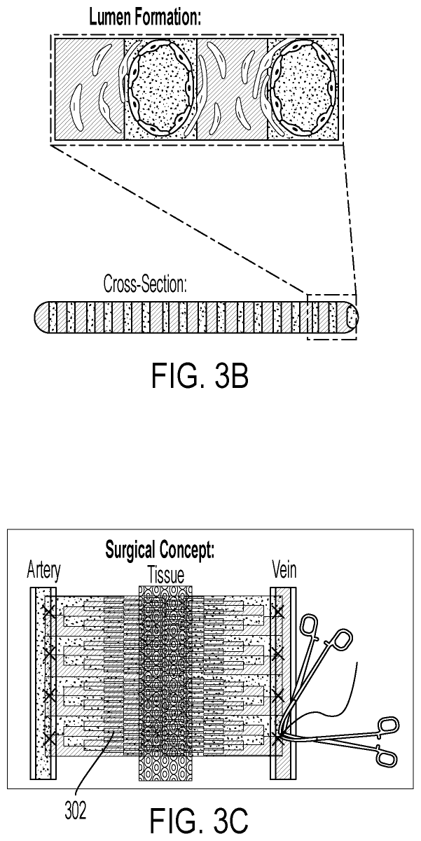

FIGS. 3A-3C illustrate an example Microvascular Appendage Sheet (MAS) printing system 300 that is capable of high resolution bioprinting of sheets 302. For example, the MAS printing system is capable of bioprinting eight 640-micron channels to 256 10-micron channels. The ability to produce a microvascular branching hierarchy that spans millimeters to single microns provides a continuous transition between capillary networks and native, microsurgically manipulatable small-diameter vessels in engineered tissues and organs. In particular, FIG. 3A shows two bioinks that are mixed into varying layer number in separate printheads. The multiplexer 200 determines which mixture 104 or 106 is sent to a nozzle 304 to control layer number and thickness in the resulting sheet 302. The nozzle 304 fans the bioink mixture (104, 106) into a wide sheet. FIG. 3B shows a MAS cross-section and that during the in vitro prevascularization stage, supporting cells such as pericytes can surround newly formed endothelial cell lumens to provide chemical and mechanical cues. FIG. 3C shows that after prevascularization, the MAS can be sutured to native vessels and wrapped around tissue to provide new blood supply.

Thus, the MAS printing system 300 is configured to chaotically print wide sheets 302 (i.e., length×width>>height) with internal layers as one continuous extrusion. As one solid construct, these sheets 302 have improved structural integrity and faster fabrication times compared to sheets traditionally bioprinted with linearly deposited cylindrical fibers. In addition, their structure and dimensions could be beneficial for integrating grafts and native vessels via microsurgery (i.e., suturing one side to a small-diameter artery, wrapping around a construct, and suturing the opposite side to a small-diameter vein).

The adjacent internal layers produced by Chaotic Printing can include bioinks of two or more cell types, such as endothelial cells and supporting mural cells, either initially homogenized into the extruded hydrogels or later perfused into vacant layers left behind by a fugitive hydrogel. The sheet could also be coated in an additional cell-laden material to include three or more cell types. Pre-implantation, cellular organization into hierarchically branching microvascular networks could be promoted in vitro by integrating pro-vasculogenic and/or pro-angiogenic growth factors, such as PDGF and VEGF, and even stimulus-respondent release mechanisms allowing for spatiotemporal guidance of vessel formation.

A Continuously Extruded Variable Internal Channeling CEVIC Device

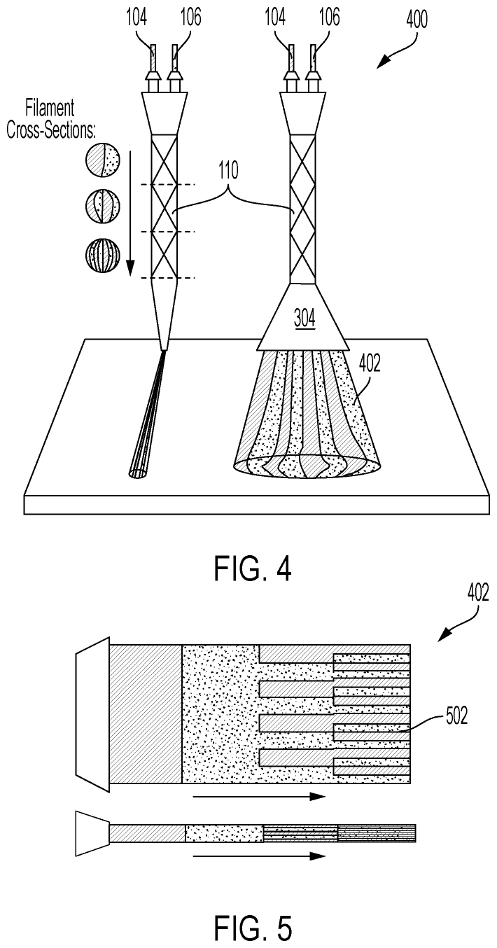

With reference to FIGS. 4-7, there is shown aspects of a CEVIC device 400 in accordance with the present disclosure. The CEVIC device achieves novel microvascular patterns that improve upon those provided by chaotic printing and MAS printing described above. As shown in in FIG. 4, the device extrudes hydrogel sheets 402 instead of filaments 118 while maintaining the alternating adjacent channeling structure (shown generally as 502) unique to chaotic printing. These channels 502 may create both cell-seeded microvascular structures or for “fugitive”/“sacrificial” inks that set up vacated spaces for nutrient inflow and waste product removal. The CEVIC device 400 allows a complete micropatterned construct covering a relatively large area (e.g., 25-300 mm²) to be produced in one extrusion, rather than requiring many passes of filament deposition.

FIG. 4 shows a kenics static mixer (KSM) 110 to produce chaotic advection during extrusion of cylindrical filaments with internal channels consisting of two or more alternating hydrogel materials. The number of KSM elements 112 (e.g., blades that divide the incoming hydrogel into twice the number of input layers) determines how many channels are found in the resulting filament. Fanning of the output produced by the nozzle 204 creates a wide sheet 402 while maintaining the alternating internal channel structure 602, producing a workable micropatterned construct in one extrusion.

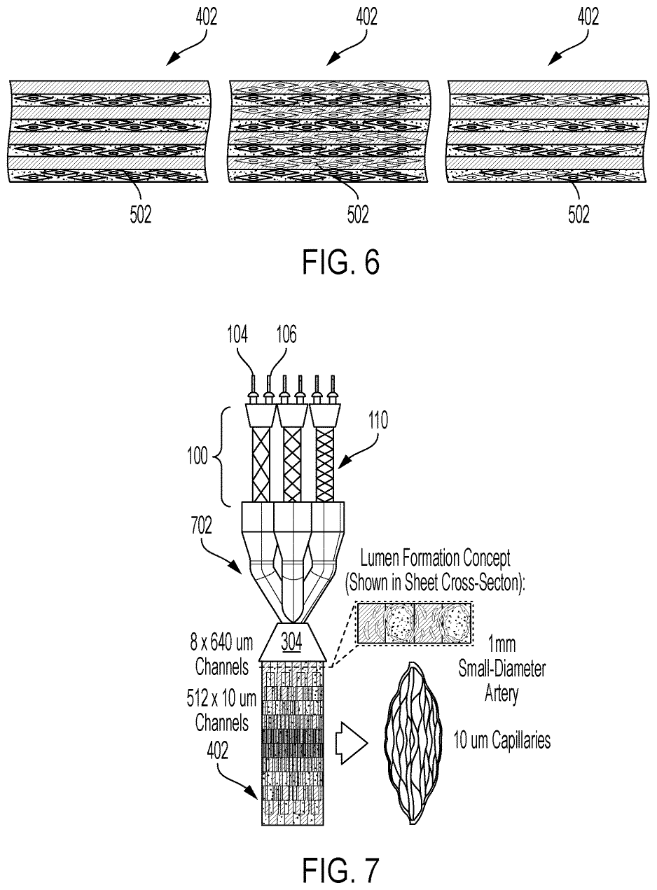

Additionally, as shown in FIG. 5, the CEVIC device 400 can switch between materials/bioinks and kenics mixers mid-extrusion to create variations in bioink type, as well as channel number and thickness throughout a continuous sheet 402 or filament extruded from a single printhead. These channels 502 can provide a basis for inducing cell alignment, positioning two or more cell types in adjacent striations, and allowing perfusion of blood or nutrient media through the tissue construct (See, FIG. 6). In particular, microchannels 502 can house one or more cell types in multiple combinations to promote cell alignment, heterocellular interactions, and/or perfusion, depending on the desired application This capability also allows the device 400 to produce hierarchically branching internal channels from artery-scale to capillary-scale, creating potential for complex, heterocellular microvascular construct fabrication (See, FIG. 7). The CEVIC device printhead is capable of producing a hydrogel sheet 402 with hierarchically branching channels 502 from artery to capillary-scale, a design concept currently being explored as a template for potentially culturing microvascular tissue constructs. The device can be used for both handheld and fully automated stage bioprinting.

Thus, the CEVIC device 400 also provides for sheet-based extrusion bioprinting for applications outside of in situ bioprinting for wound repair. Additionally, the CEVIC device 400 extends chaotic printing to sheet-based extrusion bioprinting, rather than filament deposition, while modulating channel number and thickness mid-extrusion throughout a chaotically printed construct for the purposes of replicating the hierarchically branching pattern of native microvasculature with accurate dimensions.

### Operational Overview

With reference to FIGS. 7-10, a more detailed description of the CEVIC device 400 will now be provided. The device 400 may include two or more Kenics Static Mixer (KSM) printheads 100. Each printhead 100 is connected to sources of two or more cell-laden hydrogel bioinks (104, 106). With the influence of a pressure source, such as an air pump (e.g., such as 102), these bioinks 104, 106 are mixed together within the KSM printheads 100 into alternating layer patterns of each material. Each KSM printhead 100 has a different number of KSM mixing elements 112, meaning that the bioinks develop a different number of alternating internal layers 502 downstream depending on what KSM printhead 100 they were initially extruded through. The layered bioinks then continue traveling to a rotary selector valve 702. This valve 702 can control what layered bioink continues on as the current extrusion output of the device. As a result, the device can switch from a layered bioink source containing, e.g., four alternating layers to a bioink source containing, e.g., eight alternating layers. Whichever layered bioink source is “selected” by the valve continues on to be “fanned” by the nozzle 304 into a wide sheet structure while maintaining the alternating layer pattern 502. The final product is a hydrogel sheet 402 containing alternating layers of two or more materials that sequentially divides into more and more layers with thinner and thinner widths, mimicking the natural structure of microvasculature in the body.

The sheet geometry and layer number/thickness are user-defined, but an attainable example could be a 5-mm-wide sheet containing eight 640-micron channels dividing continuously into two hundred and fifty six 10-micron channels. The bioinks can be seeded with vascular cells, such as endothelial cells and pericytes, which organize themselves into tubular vessels within the sheet's layers in a 1-2 week culture period. At this point, the microvascular sheet 402 can be later implanted into a living animal to be perfused with native blood supply and direct this blood supply to specified tissues.

KSM Printheads, Static Valve, and Perfusion Chamber Creation.

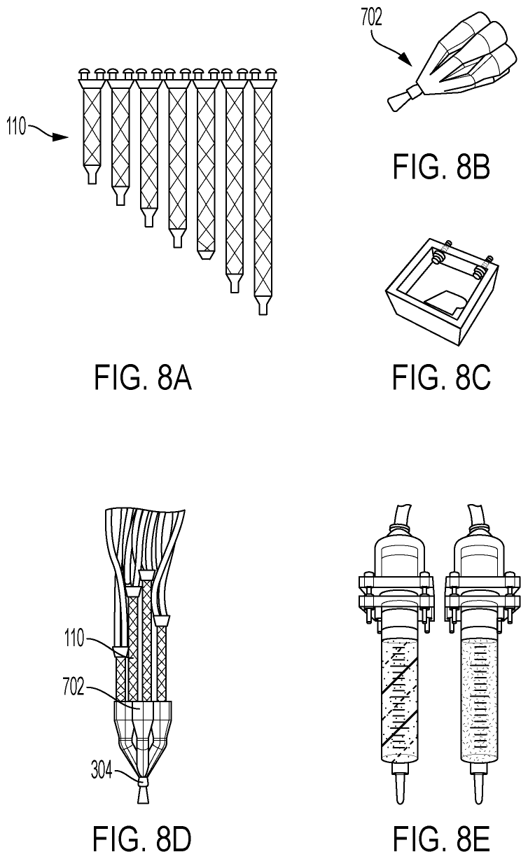

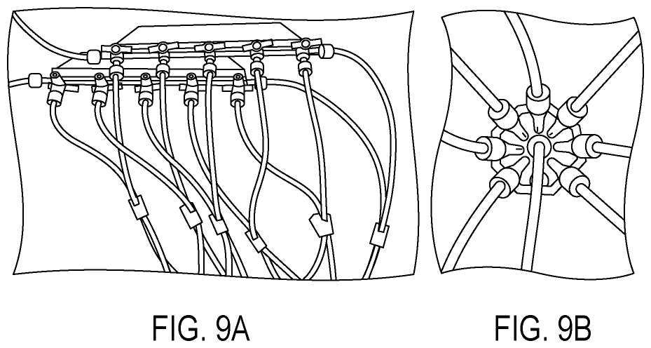

FIGS. 8A-8E show example 3D printed components of the CEVIC device. FIG. 8A shows the Kenics static mixers (KSMs) 110 with 3-9 mixing elements to produce 8, 16, 32, 64,128, 256, and 512 channels, respectively. FIG. 8D shows the KSMs are positioned in 7-input static valve 702 that directs selected flow pattern through the fanning nozzle 304 outlet to produce a sheet 402, by hand or fixed to a 3D printer (see, FIG. 8C). FIG. 8E shows the components to maintain air-tight seal between air pump and syringes containing hydrogel inks.

The 3D printing may be performed beginning with a kenics static mixing (KSM) printhead STL file edited with Autodesk Meshmixer (San Francisco, CA, USA) computer-aided-design (CAD) software. The 7-input static valve 702 printhead shown in FIG. 8B and printhead holder piece of FIG. 8C may also be designed using SolidWorks CAD software (Waltham, MA, USA). Each input to the printhead was modeled to fit one of the seven KSMs, as shown in FIG. 8D. Components were also designed to couple the pneumatic air source to the hydrogel/bioink syringes (see, FIG. 8E). A fan-shaped (i.e., oblong, 5 mm width by 0.5 mm height) nozzle design was combined to the output of the static valve printhead in MeshMixer CAD software from Autodesk (San Francisco, CA, USA).

All parts may be exported as .STL files and imported into Envision Tec (Dearborn, MI, USA) Perfactory RP software. This software positioned the . STL files on the buildplate, built supports, and produced job files for use on a Perfactory P3 DLP 3D printer. Job files were uploaded to the printer using Perfactory Observer software and the parts were printed with E-Shell 300 and E-Clear resins from EnvisionTec. After printing, each part was washed with a sequence of isopropanol, acetone, ethanol, and deionized (DI) water before drying in a dessicator. Parts were then post-cured for 40 minutes in a 3D Systems (Rock Hill, SC, USA) Procure 350 UV light box.

### Example Device Set-Up

With reference to FIGS. 9A-9B, two 20-mL syringes were filled with hydrogels of choice (i.e., depending on test of interest) slightly past 20 mL, tapped vertically to dislodge any bubbles, and pushed to the 20 mL to remove the bubbles. Each syringe is then connected to the inputs of either of two manual channel valves, as shown in FIG. 9A for manual mixer selection. FIG. 9B shows two electric rotary valves from Aurora Pro Scientific (Midland Park, NJ, USA) for automated mixer selection using two 1/16″ ID rubber tubing of equal length. Next, enough 1/16″ tubing of equal length were cut to connect each valve output to one of the two inputs on a respective KSM, so that each mixer has two different inputs (e.g., fourteen tubes for seven KSMs). The syringes in the valve-syringe apparatus were then secured with the coupling components to a MAC100Q air compressor (i.e., pump) (Makita, Anojo, JP).

The seven KSMs were each fitted with a ¼″ long piece of ⅛″ ID rubber tubing at their outlet to form a water-tight seal in the seven-input static valve printhead. They were then inserted securely into the static valve printhead so that the inlets of the 3, 5, 7, and 9-element KSMs were parallel to the extrusion plane of the fanning nozzle outlet, while the 4, 6, and 8-element KSMs were inserted with their inlets perpendicular to the extrusion plane of fanning outlet nozzle. This arrangement allowed all channels to be extruded on the same plane despite the sequential 90° axial rotation of each mixing element within the KSMs.

### Example Device Operation

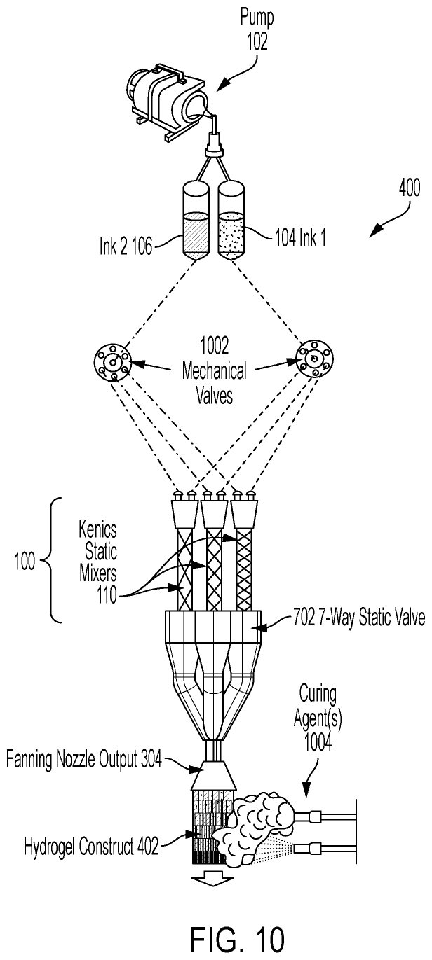

FIG. 10 illustrates additional operational details of the example CEVIC device 400, FIG. 10 shows electric rotary valves 1002 for automated input selection. Mechanical valves 1002 control which kenics static mixer 110 receives the two ink inputs 104, 106. The chosen mixer creates a certain number of alternating channels 502 of the two inks within the volume that exits the nozzle output 304 to form a sheet 402, which is then solidified via curing agent(s) 1004.

Because varying the number of kenics mixer elements 112 varies the channel number and thickness, controlling the mechanical valves therefore controls channel number and thickness in the resulting sheet construct mid-extrusion.

Hydrogel Printing. The pump 102 is turned on to pass the hydrogels 104, 106 through both syringes at a 1 mL/min flow rate, with 0.5 bar supplied to each syringe, to first fully saturate the apparatus. Each KSM 100 is checked visually for air bubbles, which are removed by tilting the seven-input static valve printhead upward while hydrogel is flowing through. Once the whole apparatus is saturated with hydrogel, the printhead is fit into the holder piece on a robotically controlled 3D printing apparatus developed in previous work in our lab and controlled by Lab VIEW from National Instruments (Austin, TX, USA).

The pump 102 is then started again while the basement is simultaneously moving at a constant 3.3 mm/s, a speed calculated to match the 1 mL/min flow rate and thereby produce uniformly wide sheets with consistent channels. For handheld printing, the basement was kept in place while the printhead was guided by hand. During manual mixer selection, the stopcocks on the manual valves were manually switched closed on each outlet at uniform intervals in order to direct the flow of hydrogel from one kenics mixer to another midflow. For automated mixer selection, two rotary valves 1002 can be programmed via Lab VIEW (or other software) to switch after a designated period of time between corresponding tubes leading to each kenics mixer.

Post-Print Curing. Immediately after extrusion, the channeled hydrogel sheet can be cured/solidified. With UV-sensitive hydrogels, such as Gelatin Methacryloyl (GelMA), the sheets are exposed to 365-nm UV light from an Omnicure S2000 (Laude, GR, NL) for 30 seconds. For hydrogels that physically crosslink in the presence of Calcium Chloride (CaCl2), such as Sodium Alginate (SA), the sheets are sprayed with a fine mist of 4% (wt./vol.) CaCl2 using either a handheld sprayer bottle or an ultrasonic atomizer. Applying CaCl2 in the form of gradually increased misting intensity allows the gel to be crosslinked with minimal impact on physical gel structure, as previously demonstrated. After waiting approximately 30 seconds, CaCl2 droplets are then added to the sheet with a pasteur pipet to completely soak the sheet. At least 5 additional minutes are then allowed to ensure complete crosslinking before handling the sheets. If the hydrogel contains both GelMA and SA, the UV and CaCl2 crosslinking steps are completed in succession.

Combining with MEW Biotextile Scaffolds. The hydrogel sheet constructs produced with this device can be combined with woven thermoplastic polymer fiber scaffolds produced by Melt Electrowriting (MEW) to combine properties of both materials. This is accomplished simply by extruding directly over a MEW scaffold before conducting the post-print curing. This allows hydrogel to crosslink around the MEW scaffold, effectively fusing both together. 500 μm polycaprolactone (PCL) scaffolds were produced with a MEW device using previously defined biotextile fabrication methods.

### Construct Fabrication Testing

Hydrogel Preparation. Sodium Alginate (SA) was added to Dulbecco's phosphate-buffered saline (DBPS) from Gibco (Billings, MT, USA) at 4% (wt./vol.) and mixed at 70° C. until fully dissolved. 3-5 drops of FluoSpheres polystyrene microspheres from Invitrogen (Waltham, MA, USA) were added to half of the prepared 4% SA, while the other half was kept clear. The final solutions were maintained at 37° C. until use. To create a “fugitive ink” (a.k.a. “sacrificial ink”), as in previous chaotic printing work, a 0.8% (wt./vol.) solution of hydroxyethyl cellulose (HEC) was prepared. HEC was mixed in DI water at 80° C. until fully dissolved, then maintained at 37° C. until use.

Channel Visualization and Measurement. For visualization purposes, 4% SA with fluorescent microspheres and 4% SA without microspheres were used as the two hydrogel inputs in the device to produce hydrogel sheet constructs with alternating clear and fluorescent channels. A 365-nm UV light was shined on the resulting hydrogel sheets to illuminate the fluorescent layers while pictures were taken with a Plugable USB Digital Microscope (Redmond, WA, USA). ImageJ software (NIH) was used to measure the width and thickness of the hydrogel sheet constructs, as well as channel thicknesses, with six replicates for each respective measurement.

Perfusion Testing. To create hydrogel sheet constructs with vacant inner channels, 4% SA with fluorescent microspheres and 0.8% HEC were used as the two hydrogel inputs in the device. During the CaCl2 crosslinking process described in section 2.3.2, SA solidified while HEC remained fluid as a fugitive ink. Resulting constructs were soaked in DI water overnight to allow the HEC to diffuse out, leaving behind vacant internal channels. A 1-mL insulin syringe was then used to inject vacant channels with orange colored DI water to demonstrate channel perfusion.

### HUVEC Viability Testing

Sterile Hydrogel Preparation. A 4.5% (wt./vol.) GelMA, 4% SA, and 0.1% lithium phenyl-2,4,6-trimethylbenzoylphosphinate (LAP) solution was prepared in DPBS to serve as a base for the cell-laden bioink. LAP powder was first constituted in DPBS and passed through a 0.22 μm filter via syringe for sterilization. Lyophilized GelMA and SA powder were exposed to UV-C light for 15 minutes before being added to the LAP solution in a sterile biosafety cabinet. The solution was mixed in sterile conditions at 70° C. until fully dissolved, then maintained at 37° C. until use.

Cell Preparation. EndoGRO Human Umbilical Vein Endothelial Cells (HUVECs) were obtained from Millipore Sigma (Burlington, MA, USA) at passage 1. Cells were thawed, seeded in two T175 flasks at 500,000 cells per flask, and incubated at 37° C. and 5% CO2 in EBM Endothelial Cell Growth Basal Medium and SingleQuots Supplements and Growth Factors from Lonza (Walkersville, MD, USA), with media changes every 72 hours. Once the flasks reached approximately 80-90% confluence, the cells were washed three times with phosphate-buffered saline (PBS) (Gibco) and detached with TrypLE (Gibco). The resulting cell suspension was pelleted down by centrifuging at 1200 rpm for 10 minutes and then aspirating the remaining media. Cells were then reconstituted in fresh media. 10 μL of the cell suspension was combined with 80 μL of PBS and 10 μL of TrypanBlue (Gibco) before being counted with a hemocytometer.

Based on the measured cell density, the suspension was transferred to a new centrifuge tube so that there were 20 million cells in the tube. Media was aspirated and 10 ml of 4.5% GelMA 4% SA 0.1% LAP hydrogel was added to the pellet and aspirated up and down to homogenize the cells within the hydrogel at a density of 2 million cells/mL. The pipet tip used at this step was trimmed approximately ⅓rd the distance from the tip to facilitate transfer of the relatively viscous hydrogel and minimize shear stress on the cells.

Bioprinting. 4.5% (wt. /vol.) GelMA 4% SA 0.1% LAP with HUVEC cells and 4.5% GelMA 4% SA 0.1% LAP without HUVEC cells were used as the two hydrogel inputs in the device for the experimental group, while 4.5% GelMA 4% SA 0.1% LAP with HUVEC cells was used for both inputs to create a “solid” control group without channels (i.e., HUVECs homogenized across the entire sheet). Sterile bioprinting was conducted by manually dragging the printhead across a well plate lid within a biosafety cabinet. Printhead components were sterilized with autoclaving and ethanol soaks, where appropriate. The kenics mixer with four mixing elements was chosen to create HUVEC-laden hydrogel sheet constructs with approximately 16 alternating channels (i.e., 8 channels of cell-laden gel alternating with 8 channels of no-cell gel). The constructs were cured within the biosafety cabinet using the UV and CaCl2 methods described in section 2.3.2. To prevent CaCl2 mist from entering the biosafety cabinet air flow, it was sprayed over the constructs within a folded enclosure of sterile aluminum foil. Printed constructs were cut into 1-cm segments with a razor blade before being placed in 24-well plates containing supplemented endothelial cell growth media (Lonza). The well plates were incubated at 37° C. and 5% CO2 for 1 week, with media changes every 72 hours.

Calcein AM Fluorescent Microscopy. Calcein AM fluorescent dye from Invitrogen (Waltham, MA, USA) was used to determine HUVEC viability, morphology, and orientation in 16-channel experimental and solid control hydrogel sheet constructs printed with the device. On days 1, 3, and 7, triplicates from each group were washed three times with DPBS before being submerged in a 2 μM solution of calcein AM in DPBS for 45 minutes at room temperature in the dark. The samples were then washed three times with DPBS and imaged using a Cytation 5 Multi-Mode Reader from BioTek (Winooski, VT, USA) at 395-nm excitation and 509-nm emission. Fixed samples were used as negative controls to account for background fluorescence.

Anti-CD31 Confocal Microscopy. WM-59 monoclonal CD31 antibodies conjugated with SuperBright 436 were used to verify CD31 protein expression, a commonly used endothelial cell marker. On day 7, six replicates from each group were washed three times with PBS and fixed with 4% (wt./vol.) paraformaldehyde for 10 minutes in an incubator at 37° C. and 5% CO2. The 4% paraformaldehyde was apirated from each sample and they were again washed three times with PBS. Each sample was then incubated with 0.1% (wt./vol.) Triton X-100 from MP Biomedicals (Santa Ana, California, USA) for 15 minutes to permeabilize the cell membranes. Samples were washed three more times with PBS before soaking in 2% (wt./vol.) bovine serum albumin (BSA) from Fisher Scientific (Waltham, MA, USA) at room temperature overnight to block excess protein binding sites. 2% BSA was removed before adding the anti-CD31 antibody solution diluted in 500 μL of 0.1% BSA to each well in the dark, leaving the plate in a 4° C. refrigerator covered in foil overnight. The next day, the antibody solution was removed, and samples were washed three more times with PBS. Samples were finally stored in PBS at 4° C. covered in foil until confocal microscopy.

Samples were imaged using a 25X objective on an Olympus (Shinjuku, Tokyo, Japan) Multiphoton FV1000 microscope, with a 405-nm excitation laser and 436-nm emission channel.

### Hydrogel Rheology

All hydrogels utilized in this study were subjected to low-viscosity flow tests (i.e., frequency sweeps) on an Anton Paar (Ashland, VA, USA) MCR 102e Modular Compact Rheometer. Viscosity measurements were collected on Anton Paar's RheoCompass software for each hydrogel between 1 1/s and 100 1/s shear rates.

### Verification of Fabrication Capabilities

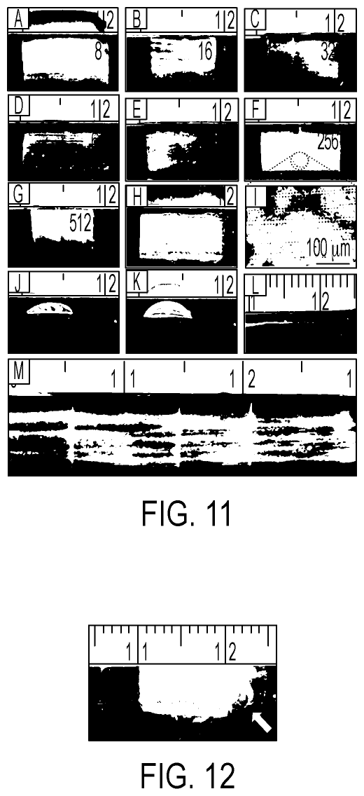

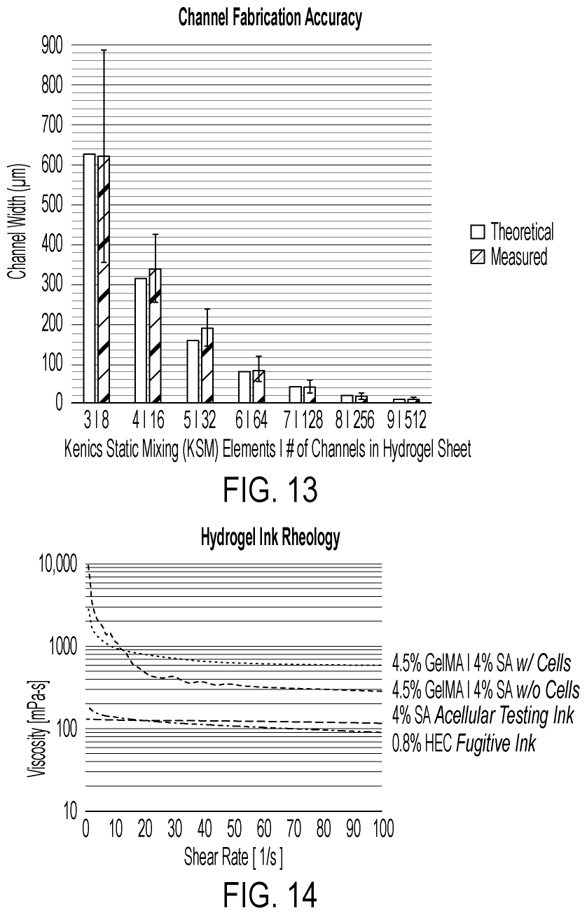

Channeled Sheet Production and Dimensional Accuracy. The CEVIC device 400 was shown to successfully extrude adjacent, alternating channels of two hydrogel inks within sheet geometries. As shown in FIG. 11, panels A-G, and FIG. 12, sheets were fabricated containing 8, 16, 32, 64, 128, 256, and 512 alternating internal channels, with average channel widths ranging from 621.5±42.92% μm to 11.67±14.99% μm, respectively. There was no trend in relative standard deviations across varying channel thicknesses. As shown in FIG. 13, the measured channel thicknesses were relatively close to their “theoretical” thicknesses (e.g., an 8-channeled 5-mm sheet should average 625-micron-thick channels). The average percentage error between measured and theoretical channel thicknesses was calculated to be 10.74%. The smallest channel thickness was measured as 10 microns within a 512-layered sheet. It's possible that smaller channel thicknesses could be achieved in future work. These constructs demonstrate the CEVIC device's ability to pattern alternating channels into sheet constructs at resolutions in the range from surgically manipulatable small-diameter arteries and veins (i.e., ~1-6 mm) to microvasculature (i.e., <100 μm) down to capillaries (i.e., ~10 μm). Channel orientation was also switched between vertical and horizontal by rotating the fanning outlet 90 degrees (FIG. 11, panels J, K). This capability could allow for multi-cellular, multi-layered sheets of tissue (e.g., skin, organ walls) with a gradient of layer patterning to be produced with a single extrusion.

FIG. 11, panel L, shows mid-extrusion material switching from a single printhead was also demonstrated with traditional filament extrusion. FIG. 11, panel M, shows additionally, mid-extrusion pattern switching was demonstrated by transitioning from 8, to 16, then 32 channels within a continuous hydrogel sheet. This capability can allow for a single, surgically manipulatable hydrogel sheet to contain complex micropatterning reminiscent of natural tissue structures such as the hiearchically branching microvascular tree, a well-documented challenge for bioprinting. The transition lengths between 8, 16, and 32-channel regions achieved with manual valve switching were approximately 1 cm. Complete automation of valve switching, as well as reducing flow rate, should allow for shorter transition lengths to be obtained. This is the subject of an ongoing study.

Sheet dimensions were measured on average to be 4.78±0.07 mm wide by 1.22±0.33 mm thick. Sheets tended to be thickest in the center, bloating beyond the 0.5 mm outlet thickness, while thinning to their edges. However, this variation was likely driven by wetting between the hydrogel and polystyrene well plate lid being used as a printing surface for demonstration purposes. Sheets with more uniform thickness could be produced with more rapid crosslinking upon deposition (e.g., UV and/or CaCl2 exposure while printing takes place) or by using a printing surface with higher affinity for the hydrogel. Early tests of extruding sheets directly into bulk CaCl2 solutions produced more consistent thicknesses (not without expected elastic swelling inherent to extruding hydrogels into crosslinking solutions) but were more prone to disrupted flow. This justifies the method of depositing sheets before subjecting them to curing. Other existing strategies for improving structural stability, such as gelatin embedding, could be integrated with the CEVIC device in future studies.

Producing Perfusable Channels with Fugitive Ink. Sheets were also produced with HEC fugitive ink that diffused away to leave vacant channels in between solid hydrogel channels. Orange-dyed water injected into vacant channels was able to exit the opposite end of the sheet, demonstrating their perfusability. Not every vacant channel was shown to be perfusable with this method, likely due to channel sinking, inexact needle placement, and variable flow rate. However, even vacant channels that aren't fully perfusable immediately upon fabrication could provide a framework for in vitro development of perfusable, endothelialized microvascular structures.

Integration with Melt Electrowriting. FIG. 11, panel H, shows PCL MEW scaffolds and 16-channelled hydrogel sheets were successfully fused into single constructs. While MEW has been combined with extrusion-based and inkjet bioprinting in past work, this is the first study combining MEW with chaotic printing, as well as with extrusion-based hydrogel sheet printing. Combining the two technologies allows for the tuned extracellular environment of hydrogels to be mechanically reinforced by the melt electrowritten woven fiber network. Using the CEVIC device to deposit a channeled hydrogel sheet over a prefabricated MEW scaffold allowed the uncured hydrogel to first penetrate the MEW scaffold pores before the curing process effectively bound the two materials together. The uncured hydrogel was drawn into space within the MEW pores via capillary action between the hydrogel and PCL fibers. With larger pores (e.g., 1 mm), this disturbed enough hydrogel volume to disrupt channel structures within the deposited sheet. However, sheets deposited onto MEW scaffolds with 350-μm pores or less maintained their channel patterning until curing. We are working to combine CEVIC and MEW printheads on one robotically controlled 3D printer to allow production of hybrid constructs within a single fabrication period.

Hydrogel Rheology. Viscosity measurements across varying shear rates for the hydrogel inks utilized in this study are presented in FIG. 14. All inks demonstrated shear-thinning behavior, an important ink characteristic for extrusion-based bioprinting to allow flow through the printhead with minimum shear stress on cells. The 4% (wt./vol.) SA and 0.8% HEC inks used for acellular testing had relatively comparable viscosities across all shear rates, justifying the choice of 0.8% as a concentration for HEC to be extruded adjacent to 4% SA to produce perfusable channels alongside SA channels. The 4.5% GelMA—4% SA ink generally had an increased viscosity at most higher shear rates with the introduction of cells but was still able to successfully produce sheets with adjacent channels of cell-laden and no-cell inks, respectively.

### Verifying HUVEC Viability

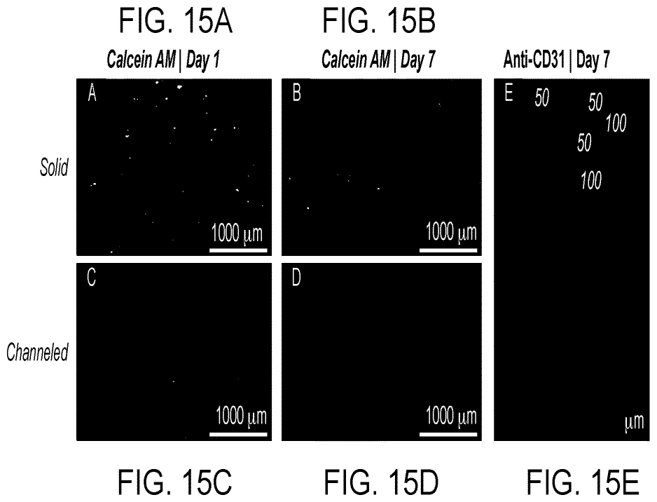

Cell Viability and Proliferation. FIGS. 15A-15D show calcein AM fluorescent microscopy images prove the HUVECs survived the CEVIC device novel printing process and maintained viability in the hydrogel ink formulation throughout the 1-week culture period. These results show that the CEVIC device novel printing process can be used effectively to bioprint hydrogel sheets containing living endothelial cells for in vitro vascular experiments and development. Hydrogel sheets with channels were observed to maintain the intended channel structure upon printing and throughout the 1-week culture period. Cells did not appear visually to migrate or proliferate significantly over the course of 7 days, but cell number was not a focus of this preliminary test and therefore not quantified. Cell seeding density was likely too low in this study for significant cell interaction. To efficiently induce cell expansion and organization into intended functional tissue structures in CEVIC constructs, future studies will need to carefully consider hydrogel pore size, cell seeding density, media components, and, potentially, interactions between two or more cell types.

Endothelial Activity. As shown in FIG. 15E, CD31 protein expression in the constructs was observed at day 7 with a fluorescently conjugated CD31 antibody under an Olympus Multiphoton FV1000 microscope, verifying endothelial expression throughout the 1-week culture period. This Z-stack data provides proof-of-concept for 3D cell morphology visualization that can help assess the structure and function of microvascular constructs tested in upcoming work.

Yet further, the device may create a sheet where no cells are included. Here, cell signaling molecules may be provided in the walls of vacant channels within the sheet. This approach allows for cells to be recruited from the host after implantation.

### Conclusion

Thus, the present disclosure presents an example CEVIC device using extrusion-based 3D bioprinting to produce wide sheet constructs for applications other than in situ wound repair.

Additionally, the CEVIC device successfully pairs this novel sheet extrusion method with the extremely fine channel-printing capability of chaotic printing. Chaotically printed filaments with adjacent internal channels have been shown to induce unidirectional cellular alignment in previous work. Thus, the CEVIC device can produce sheet constructs mimicking the shape of thin tissue layers for a wide variety of applications that require cellular alignment (e.g., neural, skin, muscle, bone, tendon, cartilage, ligament, cornea, vascular, etc.) with precise control over the thickness of each cell channel. These sheet constructs can include hundreds of adjacent cell channels as thin as a single cell that are inherently fused together with one continuous extrusion. This is in contrast to filament-based extrusion bioprinting that would require hundreds of filament passes and successful fusing between each filament to produce a construct with the same width and channel resolution.

The CEVIC printhead can deposit unidirectional channels into a vat for subsequent light-assisted bioprinting of complex construct designs. These constructs would contain one or more cell types following an intended alignment path, bringing the capabilities of chaotic printing to light-assisted bioprinting for the first time. Additional channeled, chaotic printed constructs can be created that include new biologically relevant geometries other than flat sheets, such as curving surfaces, hollow tubes, and solid organs.

The complex gating system of as many as 14 inputs to the CEVIC device printhead is able to start with two simple syringes driven by an air pump or syringe pump. As such, the CEVIC device could be merged with existing commercially available bioprinters and/or biofabrication devices to combine their capabilities. One such device is a MEW printer, wherein the CEVIC device produced sheet constructs that can fuse to MEW biotextile scaffolds. This produces composite sheet constructs with enhanced material strength that can be handled and sutured as an implant during future in vivo studies. Additionally, the CEVIC device printhead can be operable by hand, providing the potential for in situ bioprinting applications, such as depositing a multi-material skin graft containing aligned cells directly over a wound site.

The present disclosure also presents a novel method of switching bioink inputs and/or micropatterning mid-extrusion that can either be controlled manually via mechanical valves or fully automated via electronically controlled valves as part of a printing sequence. The CEVIC device can switch the number and thickness of channels mid-extrusion, from the mm-scale of structures such as small-diameter blood vessels to the 10 μm-scale of capillaries, in continuous succession. The CEVIC device can thus produce a hiearchically branching network of microvasculature-like channels across length scales not yet achieved in other microvascular fabrication strategies. The present disclosure also proves the viability and continued vascular expression of endothelial cells seeded in sheet constructs for at least a week, suggesting the potential of CEVIC device constructs for developing microvascular tissue constructs. Furthermore, the use of a fugitive ink has been demonstrated to create perfusable vacant channels that could potentially guide development of functional microvasculature in cell-seeded sheet constructs. These constructs could eventually be applied as implants to promote vascularization in wound sites, microvasculature supply to larger tissue grafts or biofabricated graft constructs, or as tissue models for drug screening and disease modeling.

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A printing device, comprising:
a plurality of input valves that receive print material;
a plurality of Kenics Static Mixer (KSM) printheads that each receive the print material from the plurality of input valves, each of the KSM printheads having a predetermined number of mixing elements that divide received print material into layers; and
a multi-way valve that directs a flow pattern to a fanning nozzle outlet,
wherein the fanning output nozzle outputs a construct as a sheet.

2. The printing device of claim 1, wherein the input valves comprise electronic rotary valves that automatically select the print material provided to the KSM printheads.

3. The printing device of claim 1, wherein the fanning output nozzle produces the sheet as a micropatterned construct in a single extrusion.

4. The printing device of claim 1, wherein the input valves switch between a plurality of print materials to create variations in the print material type.

5. The printing device of claim 1, wherein each KSM printhead has a different number of mixing elements that create a different number of alternating internal layers in the sheet.

6. The printing device of claim 4, wherein the variations comprise a channel number and thickness throughout a continuous sheet extruded from a KSM printhead.

7. The printing device of claim 1, wherein the print material is a bioink and wherein the sheet is a tissue construct used for perfusion of blood or nutrient media.

8. The printing device of claim 7, wherein microchannels are formed within the sheet to promote cell alignment, heterocellular interactions, or perfusion.

9. The printing device of claim 1, wherein the sheet is a hydrogel sheet containing alternating layers of two or more print materials that sequentially divides into more layers with thinner widths.

10. The print device of claim 9, wherein the sheet is structurally similar to microvasculature in a body.

11. The print device of claim 10, wherein the sheet has a geometry, number of layers and a thickness that is user selectable.

12. The print device of claim 1, wherein the print material is seeded with vascular cells that are organized into tubular vessels within layers of the sheet.

13. The print device of claim 1, wherein the sheet is cured using ultraviolet light.

14. The print device of claim 1, wherein the print device provides for combinations of print material by switching mid-extrusion through a single printhead.

15. The print device of claim 1, wherein the sheet is a complete micropatterned construct covering an area between 25-300 mm² that is produced in one extrusion.

16. A method of printing a micropatterned construct, comprising:
pumping a plurality of bioinks to respective valves;
using the valves to control which of the plurality of bioinks is provided to kenics static mixing (KSM) printheads;
mixing the output of the KSM printheads;
providing mixed output of the KSM printheads to a fanning nozzle; and
extruding a hydrogel sheet construct from the fanning nozzle.

17. The method of claim 16, further comprising curing the hydrogel sheet.

18. The method of claim 16, further comprising forming channels and branches within the hydrogel sheet.

19. The method of claim 16, wherein the mixing further comprises creating alternating channels in the hydrogel sheet.

20. The method of claim 16, wherein a structure of the hydrogel sheet simulates a hierarchical structure of microvasculature.
